(19) United States
(12) Patent Application Publication
(10) Pub. No.: US 2016/0235911 A1
(43) Pub. Date: Aug. 18, 2016

(54) REMOTELY ACTIVATED DRUG DELIVERY SYSTEMS, VIBRATORY DRIVE MECHANISMS, AND METHODS

(71) Applicant: Regeneron Pharmaceuticals, Inc., Tarrytown, NY (US)

(72) Inventors: Michael Cupicha, East Schodack, NY (US); Alexei Goraltchouk, Cambridge, MA (US); Mykhaylo Hrytsak, Hastings-on-Hudson, NY (US)

(73) Assignee: Regeneron Pharmaceuticals, Inc., Tarrytown, NY (US)

(21) Appl. No.: 15/042,560

(22) Filed: Feb. 12, 2016

Related U.S. Application Data
(60) Provisional application No. 62/115,285, filed on Feb. 12, 2015.

(51) Int. Cl. A61M 5/142; A61M 5/172; F04B 43/12; A61M 5/145

(52) U.S. Cl. CPC A61M 5/14228; A61M 5/14224; A61M 5/1452; A61M 5/172; F04B 43/12; A61M 2005/14506

(57) ABSTRACT

Drug delivery systems, vibratory drive mechanisms, and methods of using the systems and mechanisms are disclosed. The drug delivery system including an activation module, a vibrating mechanism electrically coupled to the activation module, a pumping mechanism connected to the activation module, a reservoir, and an injection mechanism coupled to the reservoir by at least one fluid pathway. The at least one fluid pathway extends through the pumping mechanism. The vibratory drive mechanism including an activation module, a vibrating mechanism electrically coupled to the activation module, and an actuation mechanism coupled to the vibrating mechanism. Methods of using a remotely activated drug delivery system are also disclosed.

### CROSS REFERENCE TO RELATED APPLICATION

This application claims priority benefit under 35 U.S.C. §119(e) to U.S. provisional application No. 62/115,285 filed Feb. 12, 2015, which is incorporated herein by reference in its entirety.

### FIELD OF THE INVENTION

The present invention relates generally to drug delivery systems for administering medication. More specifically, but not exclusively, the present invention concerns drug delivery systems and vibratory drive mechanisms.

### BACKGROUND OF THE INVENTION

Currently it may be difficult for elderly, young, or incapacitated patients, such as those with, for example, dementia, Alzheimer's, extreme invalids, and certain handicaps, to timely and accurately take their medications. Patients often forget to take their medication on time or take the wrong amount of medication. Even when patients do timely and accurately take their medications they may tell their doctor the wrong information regarding when they took their medication and how much medication they took. Thus, a better mechanism for delivering medication and keeping records of that medication delivery is needed.

### SUMMARY OF THE INVENTION

Aspects of the present invention provide drug delivery systems, vibratory drive mechanisms, and methods for using the drug delivery systems and vibratory drive mechanisms.

In one aspect provided herein is a drug delivery system including an activation module, a vibrating mechanism electrically coupled to the activation module, a pumping mechanism connected to the activation module, a reservoir, and an injection mechanism coupled to the reservoir by at least one fluid pathway. The at least one fluid pathway extending through the pumping mechanism.

In another aspect, provided herein is a vibratory drive mechanism including an activation module, a vibrating mechanism electrically coupled to the activation module, and an actuation mechanism coupled to the vibrating mechanism.

In yet another aspect, provided herein is a method of using a remotely activated drug delivery system, the method includes positioning the remotely activated drug delivery system on a patient. The method also includes sending an activation signal to the remotely activated drug delivery system to administer an injection. The method may further include receiving the activation signal in the remotely activated drug delivery system. The method may also include processing the activation signal to deploy an injection mechanism of the remotely activated drug delivery system to deliver a medication to the patient. Further, the method includes retracting the injection mechanism once the medication is delivery to the patient.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a top perspective view of a drug delivery system with a transparent housing, in accordance with an aspect of the present invention;

FIG. 2 is a side view of the drug delivery system of FIG. 1 with a transparent housing, in accordance with an aspect of the present invention;

FIG. 3 is a partially exploded side perspective view of an activation module and pumping membrane system, in accordance with an aspect of the present invention;

FIG. 4 is a front perspective view of the activation module and pumping membrane system of FIG. 3 at the start of activation, in accordance with an aspect of the present invention;

FIG. 5 is a side perspective view of the activation module and pumping membrane system of FIG. 3 near the end of activation, in accordance with an aspect of the present invention; and

FIG. 6 is a perspective view of another drug delivery system, in accordance with an aspect of the present invention;

FIG. 7 is a perspective view of an activation module and switching mechanism for the drug delivery system of FIG. 6, in accordance with an aspect of the present invention;

FIG. 8 is a computing environment utilizing aspects of the present invention;

FIG. 9 is a workflow diagram of one method of using the drug delivery system, in accordance with an aspect of the present invention;

FIG. 10 depicts one or more aspects of an EIR terminal utilized in an embodiment of the present invention;

FIG. 11 depicts one embodiment of a single processor computing environment to incorporate and use one or more aspects of the present invention; and

FIG. 12 depicts one embodiment of a computer program product incorporating one or more aspects of the present invention.

### DETAILED DESCRIPTION FOR CARRYING OUT THE INVENTION

Generally stated, disclosed herein are drug delivery systems and vibratory drive mechanisms. The drug delivery systems herein may be remotely activated. Further, methods of using the remotely activated drug delivery systems and vibratory drive mechanisms are discussed.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of attachment, while “distal” indicates the portion of the device farthest from the point of attachment. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

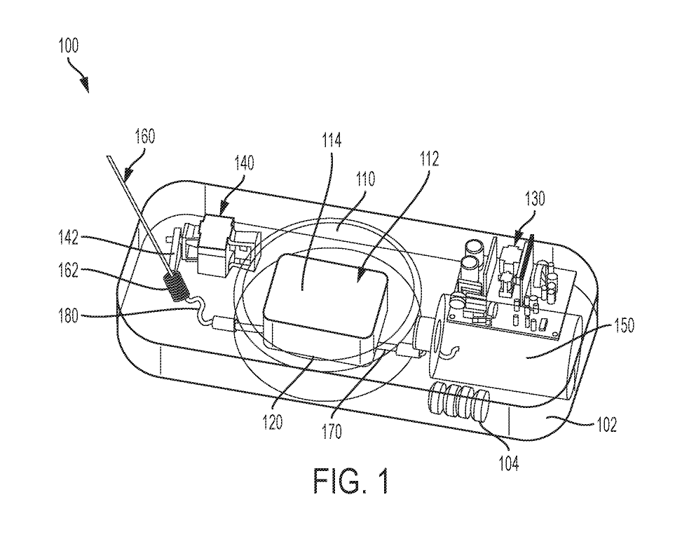

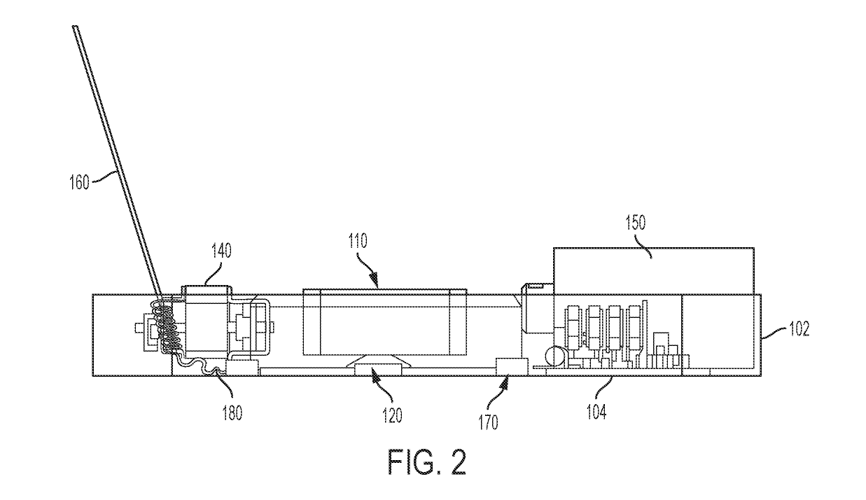

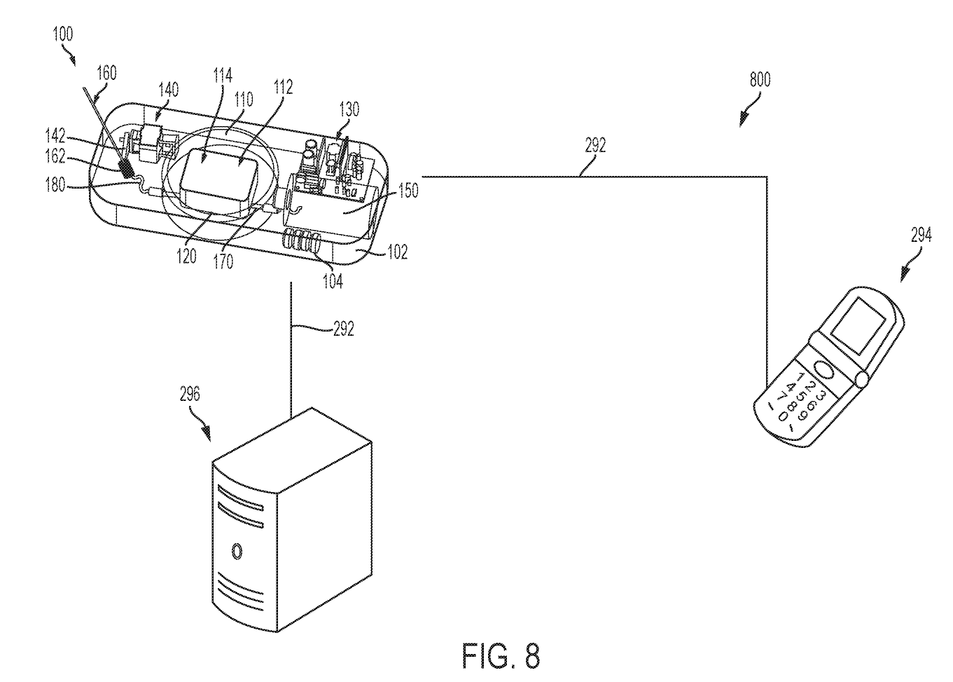

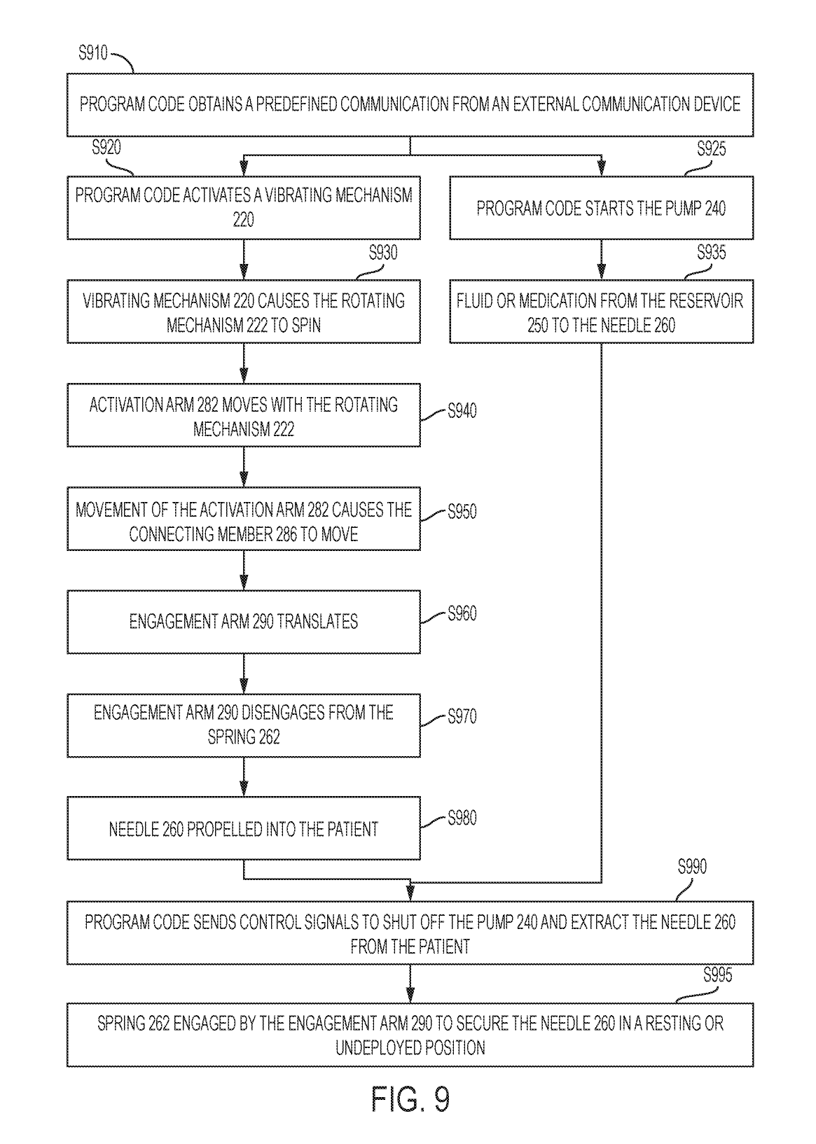

Referring to the drawings, wherein like reference numerals are used to indicate like or analogous components throughout the several views, and with particular reference to FIGS. 1 and 2, there is illustrated a drug delivery system 100. The drug delivery system 100 may be remotely activated. The drug delivery system 100 may include a housing 102, a power supply 104, an activation module 110, a pumping membrane 120, controller 130, a motor 140, a reservoir 150, and an injection mechanism 160. The injection mechanism 160 may be, for example, a needle, microneedle, cannula, flexible cannula, catheter, or the like for a subcutaneous injection or a tube, dispensing needle, or the like for topical application to the skin, a patch, or the like by dispensing a stream or drip of fluid. The power supply 104 may be positioned within the housing 102 and may include, for example, at least one battery or other power source 104. The activation module 110 includes a receiver for receiving the activation signal, for example, telephone call and/or any voice, text, or other data communication over a wired and/or wireless communications network, known to one of skill in the art, and a vibration mechanism 112 positioned within the activation module 110. The receiver in the activation module 110 may also include a transmitter (not shown) in order to communicate with external devices, such as a device sending the aforementioned communication over a network. The transmitter and the receiver may be a combined unit and/or a separate unit. The communication between the drug delivery system 100, specifically, the activation module 110 and any external devices will be further explained in the context of FIGS. 8-9.

Returning to FIG. 1, the pumping membrane 120 may be positioned near a bottom of the housing 102. The activation module 110 may be positioned over, in contact, and/or communicatively coupled with the pumping membrane 120. The activation module 110 may also be communicatively coupled to the controller 130, such that upon obtaining a communication (e.g., a phone call), the activation module 110 can communicate receipt of the communication to the controller 130. The controller 130 may also be positioned within the housing 102 and may be, for example, a printed circuit board, including processing circuit, which may also be referred to as a processor and/or a microprocessor. This processor may execute computer program code, and through this execution, communicate with and control various mechanical components of the drug delivery system 100, including but not limited to, the motor 140. The computer program code executed by the processor may reside in a memory device internal to the housing 102, for example, it may be part of the controller 130 and/or in the activation module 110. The computer program code (which may also be referred to as software) may reside on one or more memory devices external to the housing 102, but accessible to the processor, via a communications network.

As aforementioned, the motor 140, which is also positioned within the housing 102, and may be communicatively coupled to the controller 130, may be configured to control the deployment and removal of the injection mechanism 160 from a patient. The reservoir 150 may also be positioned within the housing 102 and may be coupled to the injection mechanism 160 by a fluid pathway or channel 170, 180. The fluid pathway 170, 180 may extend from the reservoir 150 passing through the activation module 110 and/or the pumping membrane 120 to couple to the injection mechanism 160 at the other end. The fluid pathway 170, 180 may be, for example, a single fluid pathway from the reservoir 150 through the pumping membrane 120 and to the injection mechanism 160. Alternatively, the fluid pathway 170, 180 may be at least two fluid pathways, for example, a first fluid pathway 170 from the reservoir 150 to the pumping membrane 120, then the fluid may be pumped by the pumping membrane 120 and out a second fluid pathway 180 from the pumping membrane 120 to the injection mechanism 160. The reservoir 150 may be, for example, a flexible container or rigid container. The flexible containers may be, for example, a fill seal, blow fill seal, or the like which assist with fluid elution as stress is applied on the flexible container. The reservoir 150 may include, for example, a means for decompressing or means for releasing pressure (not shown) as the fluid is removed or pumped from the reservoir 150 to a patient. The means for releasing pressure may be, for example, a flexible or elastic reservoir container, a vented reservoir, a pressurized reservoir, or the like. The flexible reservoir container may be of the type that deflates as fluid is pumped out. The vented reservoir would allow for air to flow into the reservoir 150 as fluid flows or is pumped out of the fluid pathway, but would also prevent fluid from flowing out of the vent. The pressurized reservoir would allow for the pressure within the rigid reservoir 150 to be adjusted as fluid flows or is pumped out of the reservoir 150. Alternative means for releasing pressure within the reservoir 150 as fluid passes into the fluid pathway 170 are also contemplated, for example, valves.

The drug delivery system 100, as shown in FIGS. 1 and 2, may be, for example, a patch pump that may be worn by the patient. In use, the system 100 would be activated by a medical professional by, for example, remotely connecting with the system 100 via a communication network to start the fluid or medication delivery. An example would be the system 100 having a designated telephone number 114 which could be called by the medical professional and/or may receive predefined data communication over a network, including at a designated port. This communication is further described in the context of FIGS. 8-9. When the system 100 receives the communication, which can include but is not limited to, a call, the activation module 110 will obtain this communication and the program code executed by the processor will obtain the communication and based on this communication, may actuate the motor 140 by rotating the arm 142 to release a spring 162 that is coupled to the injection mechanism 160. When the spring 162 is released the injection mechanism 160 is moved to a deployed position, for example, driven into the patient or positioned to release medication for topical application on a patient. In addition, when the system 100 is activated, the activation module 110 begins to vibrate on top of the pumping membrane 120. The pumping membrane 120 may be, for example, at least one of a pliable or flexible surface, which may be conical shaped, and is positioned over a rigid surface such that it forms a cavity which may be filled with air. The fluid pathway 170, 180 may extend through the cavity formed by the pumping membrane 120. In an embodiment of the present invention, the activation module 110 may separately include a processor (not pictured), which may communicate with the controller 130. In this embodiment, when the activation module 110 receives a communication, such as a call, the program code executed by the processor in the activation module 110 communicates with the controller 130 to actuate the motor 140 in the manner described above. The computer program code executed by the processor in the activation module 110 may be stored on one or more memory resources accessible to the processor via a communications connection, including but not limited to, a memory local to the device utilized to send the communication to the activation module 110.

The vibration mechanism 112 in the activation module 110 vibrates to cause an up and down movement which transmits a pumping action to the pumping membrane 120. As the pumping membrane 120 is activated the fluid or medication in the reservoir 150 is driven or pumped out of the reservoir 150, through the fluid pathway 170, 180, and through the injection mechanism 160 to the patient. As the fluid is pumped out of the reservoir 150, the means for releasing pressure (not shown) is activated to compensate for the loss of fluid within the reservoir 150. Once the programmed amount of medication from the reservoir 150 is delivered to the patient, the activation module 110 stops and the motor 140 reverses and extracts the injection mechanism 160 from the deployed position and the system 100 shuts off. Before the system 100 shuts off, it is also contemplated that the system 100 may send a report or data back to the medical professional who activated the injection cycle. The report or data may include, for example, time of injection, amount of medication, duration of injection, and the like to allow the medical professional to remotely monitor the patient's treatment schedule.

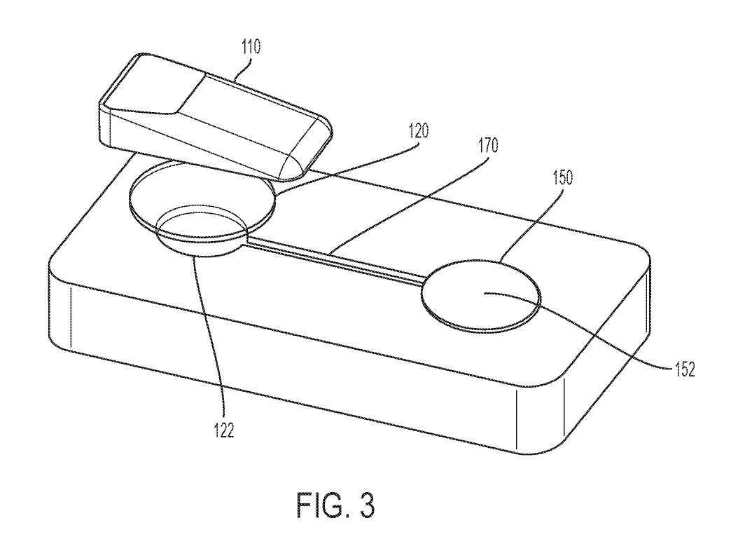

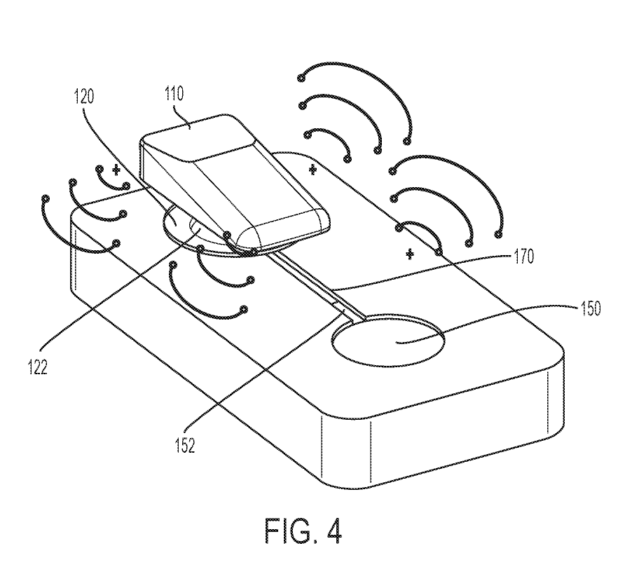

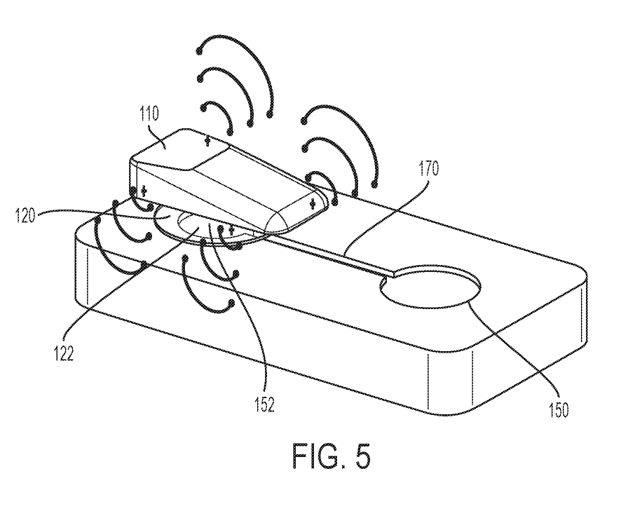

FIGS. 3-5 illustrate how the vibrating mechanism (not shown in FIGS. 3-5) of the activation module 110 may be used to pump fluid out of a reservoir 150. As illustrated, the vibrating mechanism of the activation module 110 is placed over a pumping membrane 120 with the pumping membrane 120 being secured over a cavity 122. The cavity 122 is fluidly connected to the reservoir 150 by a fluid pathway 170. Although not shown in FIGS. 3-5, the cavity 122 may also be connected or coupled to another fluid pathway to allow the medication or fluid in the reservoir 150 to pass through the cavity 122 and be administered directly to a patient. As shown in FIGS. 4-5, once the vibrating mechanism of the activation module 110 is placed over the pumping membrane 120 and activated, the vibrating mechanism begins to vibrate exerting a force on the pumping membrane 120. The force exerted on the pumping membrane 120 causes the fluid 152 in the reservoir 150 to be driven or pumped from the reservoir 150 through the fluid pathway 170 into the cavity 122.

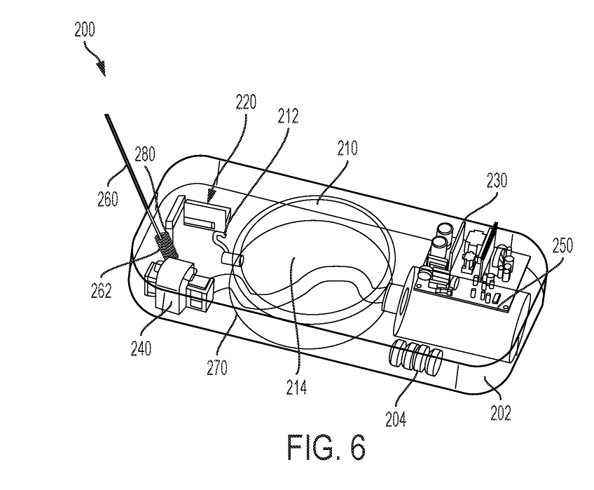

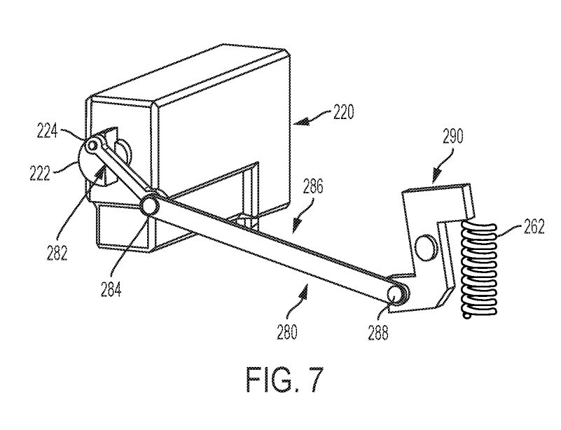

Referring now to FIGS. 6-7, another drug delivery system 200 is shown. The drug delivery system 200 may be, for example, remotely activated. The delivery system 200 includes a housing 202 with a power supply 204, an activation module 210, a vibrating mechanism 220, a controller 230, a pump 240, a reservoir 250, an injection mechanism 260, and an actuation mechanism 280. The power supply 204 may be positioned within the housing 202 and may include, for example, at least one battery or other power source. The activation module 210 includes a receiver for receiving the activation communication, from for example, a telephone call and/or data communication, and at least one processor to send a signal to start an injection using the drug delivery system 200. In a further embodiment of the present invention, the activation module 210 may also include a transmitter. As discussed above, a processor can be part of the controller 230 and/or part of the activation module 210. The vibrating mechanism 220 is positioned in the housing 202 near the activation module 210. The activation module 210 may be connected to the vibrating mechanism 220 by wires 212 or other circuitry components as known to one of skill in the art. The controller 230 may also be positioned within the housing 202 and may be, for example, a printed circuit board and may include a processor. The pump 240 is also positioned within the housing 202 and may be turned on by the controller 230 when the activation module 210 is activated. The reservoir 250 may be positioned within the housing 202 and may be coupled to a first end or inlet of the pump 240 by a fluid pathway 270. The fluid pathway 270 may be, for example, a tube, channel, vial, syringe, or the like that allows for the passage of fluid from a reservoir 250 to injection mechanism 260 and/or pumping mechanism 240. A second end or outlet of the pump 240 may be coupled to the injection mechanism 260 to allow for the fluid or medication from the reservoir 250 to be passed or pumped using at least one fluid pathway 270 to the injection mechanism 260 and to the patient. The actuation mechanism 280 may be coupled to the vibrating mechanism 220 at a first end and may engage the injection mechanism 260 at a second end.

The reservoir 250 may be of the type described above with reference to reservoir 150, which will not be described again here for brevity sake. The pumping mechanism 240 may be, for example, a peristaltic pump, a rotary pump, or the like. For example, the peristaltic pump 240 would exert a force on the fluid pathway 270 to pump the fluid in the fluid pathway 270 through the injection mechanism 260 and to the patient. Alternatively, the rotary pump 240 would receive the fluid from the fluid pathway 270 in a chamber (not shown) within the pump 240, move the fluid within the chamber to a second fluid pathway, and pump the fluid out the second fluid pathway and to the patient.

The actuation mechanism 280 is shown in greater detail in FIG. 7. The actuation mechanism 280 may include an activation arm 282, a connecting member 286, and a latch 290. A second end of the activation arm 282 may be rotatably coupled to a first end of the connecting member 286 by a first pivoting mechanism 284. The second end of the connecting member 286 may be rotatably coupled to a first end of the latch 290 by a second pivoting mechanism 288. A first end of the activation arm 282 may be secured to a rotating mechanism 222 extending out from a side of the vibrating mechanism 220. The activation arm 282 may be fixed to the rotating mechanism 222 by a fastener 224 so that as the rotating mechanism 222 turns the activation arm 282 moves with the rotating mechanism 222. The rotating mechanism 222 may be, for example, a half circle with a flat side and a curved side which meet to form a first end and an opposite second end. A second end of the latch 290 may engage a spring 262. The actuation mechanism 280 could alternatively be, for example, an on/off switch, a momentary switch, or the like that may be contacted by the rotating mechanism 222 to release the spring 262. It is also contemplated that the actuation mechanism 280 could be replaced with, for example, an electrical switch actuation mechanism 280 that would send a signal from the activation module 210 to the latch 290 to release the spring 262.

The drug delivery system 200 of FIG. 6 may be, for example, a patch pump that may be worn by the patient. To use the system 200, a medical professional or caregiver would connect to the system 200 to start the fluid or medication delivery. The medical professional or caregiver could, for example, remotely connect to the system 200 and could activate the system 200 by sending a predefined communication over a network, including but not limited to, calling a designated telephone number 214 for the system 200. The activation module 210 of the system 200 receives the call and may transmit a signal through wires 212 and/or other electronic components (and/or wirelessly) to the vibrating mechanism 220. When the vibrating mechanism 220 is activated and starts to vibrate it may cause the rotating mechanism 222 to spin. The vibrating mechanism 220 may activate the rotating mechanism 222 by vibrating the rotating mechanism 222 to an off balanced position to initiate rotation or by turning on a rotating motor (not shown) positioned within the vibrating mechanism 220 and coupled to the rotating mechanism 222 to initiate rotation. As the rotating mechanism 222 spins, the activation arm 282 moves with the rotating mechanism 222. The movement of the activation arm 282 may in turn cause the connecting member 286 to move. As the connecting member 286 moves the latch 290 will also translate. The translation of the latch 290 will cause the latch 290 to disengage from the spring 262. Once the latch 290 releases the spring 262, the spring 262, which is coupled to the injection mechanism 260, exerts a force on the injection mechanism 260 propelling the injection mechanism 260 to a deployed position for injection or administration, for example, into the patient or over the patient's skin for topical applications. Simultaneously, the activation module 210 may send a signal to the pump 240 to start the pump 240. When the pump 240 is activated, fluid or medication from the reservoir 250 is pumped into the injection mechanism 260. The fluid or medication travels from the reservoir 250 through the fluid pathway 270 to the injection mechanism 260 for delivery to the patient. The fluid pathway 270 may be a single pathway from the reservoir 250 to the injection mechanism 260 or alternatively, may be a first fluid pathway from the reservoir 250 to the pump 240 and a second fluid pathway from the pump 240 to the injection mechanism 260. Once the requested amount of fluid or medication from the reservoir 250 is delivered to the patient, the pump 240 shuts off and a signal is sent to withdraw the injection mechanism 260 from the patient or retract the injection mechanism 260 from its deployed position. When the injection mechanism 260 is retracted back into the housing 202, the spring 262 is again engaged by the latch 290 to secure the injection mechanism 260 in a resting or undeployed position.

The drug delivery system 100 of FIGS. 1 and 2 and the drug delivery system 200 of FIGS. 6 and 7 may be actuated and controlled remotely by a medical professional by several different modalities including but not limited to a telephone, blue-tooth, micro-wave, high-frequency radio, laser, infrared, or other similar technologies, without the need for patient assistance or intervention. If a telephone call is used to activate the systems 100, 200, then the systems 100, 200 may be programmed to only receive calls from a designated number and to block all other numbers to prevent inadvertent medication administrations. The drug delivery systems 100, 200 may be remotely activated from both short distances and long distances. The ability to remotely control the drug delivery systems 100, 200 allows for accurate, real time administration of a required dosage to a patient by a medical professional from another location.

Program code executed by a processor in the systems 100, 200 may also record the injection data in a memory accessible to the system 100, 200 either internally and/or over a communications network, and/or automatically transmit the injection data back to the medical professional. The injection data may include, for example, patient name, device number, injection date and time, dose amount, duration of the dose, drug injected, confirmation of completed injection, number of doses left to be administered, who administered the dose, when the next dose is due, and the like. The systems 100, 200 may also include an access point, including but not limited to an antenna (not shown) to enable the data transfer from the systems 100, 200 to a remote storage location. The access point may be built in to the systems 100, 200 and may be, for example, dome shaped, with or without signal boosters, blue-tooth compatible, and others which would allow for the injection data to be transferred. By automatically transmitting the injection data back to the medical professional, the systems 100, 200 provide for better record keeping and remove any errors in the information which the medical professional may receive from the patient due to miscommunication or forgetfulness of the patient. Program code executing on a processor in the systems 100, 200 may also encrypt the injection data from the systems 100, 200 before sending it to the medical professional. The program code executing on the processor in the systems 100, 200 may store the encrypted data on a shared external resource, such as one or more servers, and/or a cloud. The external resource may be accessible over a communications network by other computing resources, devices, including but not limited to, Smartphone, tablets, laptops, and/or personal computers. By storing the injection data on the systems 100, 200 and remotely, authorized medical professionals may access the injection data from any location, thus allowing for both the patient's treating physician from any location, as well as any emergency personnel to access the data. The remotely stored injection data could also then be downloaded by the patient's medical providers and incorporated into their electronic medical records.

The systems 100, 200 may also include a notification feature which allows the medical professional to remotely activate a patient notification module to advise the patient of when the injection will begin. The notification may be, for example, a beep or an automated message that tells the patient when the next dose will be administered. The systems 100, 200 may also include an encoded information reading (EIR) terminal, configured to read encoded indicia including but not limited to barcodes and RFIDs. In an embodiment of the present invention, the mobile housing houses an EIR terminal that can be utilized to scan the label on the reservoir 150, 250. Program code executing on the EIR terminal can decode the encoded indicia and transmit the decoded data, for example, the type of medication loaded in the system 100, 200, back to the treating medical professional.

In addition, the systems 100, 200 may be equipped with monitoring devices (not shown), such as, temperature sensors, pressure sensors, electrocardiogram sensors, blood sugar level sensors, and other like body or vital sensors, sensor modules, or devices, which allow the system 100, 200 to be activated only when the system 100, 200 is attached to the patient. The systems 100, 200 may include, for example, at least one a thermocouple that contacts the fluid or medication to sense its temperature before being delivered. If a thermocouple is used for sensing the temperature of the medication, the systems 100, 200 would be programmed so that the systems 100, 200 could only be activated if the temperature of the medication was within the desired temperature range for being administered. The systems 100, 200 may also include, for example, at least one thermocouple for contacting the patient. The thermocouples used for confirmation of patient contact would be programmed such that the systems 100, 200 could only administer medication if the thermocouples sensed the systems 100, 200 were on a surface within a defined range that correlates to the range of normal body temperatures. Alternatively, the sensors may be used, for example, to send a signal notifying the caregiver or doctor that an administration of a given medication or fluid is needed. The caregiver or doctor may then review the provided sensor data and remotely administer the needed medication or fluid to the patient.

The remote activation mechanism of the drug delivery systems 100, 200 may also be used in bed-side equipment in nursing homes and hospitals, as well as in home-care equipment stations to remotely start a test or treatment. For example, a blood pressure cuff could be remotely activated to test a patient's blood pressure without a medical professional being in the room with the patient.

It is further contemplated that the drug delivery systems 100, 200 may include additional sensors that could sense when a patient was moved from, for example, an operating room to a recovery area. The sensors would be programmed to then notify the activation module 110, 210 of a change in treatment protocol. Once the activation module 110, 210 received the change in treatment protocol the new treatment protocol would be instituted and medication would be injected into the patient based on the new treatment protocol without the need for a medical professional to administer the medication.

FIG. 8 is an example of a computing environment 800 that can be utilized by embodiments of the described systems 100, 200. Numbering references in FIG. 1 are used for simplicity, but the described computing environment 800 can be utilized with various embodiments of the present invention. As explained earlier, the drug delivery system 100 of FIGS. 1 and 2 and the drug delivery system 200 of FIGS. 6 and 7 may be actuated and controlled remotely, among other aspects, the computing environment of FIG. 8 can be utilized to control the disclosed drug delivery systems, remotely.

In the computing environment 800 of FIG. 8, the drug delivery system 100 is communicatively coupled to a communications network 292. The communications network 292, although shown as a singular network, may include a plurality of devices and communications networks in order to enable a data and/or voice communication between the drug delivery system 100 and other elements of the computing environment 800. As described earlier, the activation module 110 includes a receiver and a transmitter that enable the drug delivery system 100 to send and receive communications via the communications network 292. Also communicatively coupled to the network is at least one communication device 294, including but not limited to a cellular telephone and/or a mobile computing device, including a Smartphone. As explained in reference to FIG. 1, the communication device 294 may communicate with the drug delivery system 100 over a variety of different communication networks, including both voice and data connections, hence, the communications network 292 represents, in accordance with the different embodiments, various communication networks known to one of skill in the art. In accordance with an embodiment of the present invention, the drug delivery system 100 receives a predefined communication from the communication device 294 over the communications network 292. Upon receipt of the predefined communication, which may include, but is not limited to, a phone call from a particular number 114 and/or a specific data packet from a particular IP address, program code executing on a processor in the drug delivery system 100 actuates the described electro-mechanical components to ultimately deliver the drug in the manner described, for example, in reference to FIG. 7.

As explained in reference to earlier embodiments, the program code executed by a processor in the system 100 may also record the injection data in a memory accessible to the system 100 on an external memory device, including but not limited to an external server 296. The external server 296 may include a resource of a cloud (not pictured).

FIG. 9 is an example workflow diagram showing aspects of a method executed by embodiments of the present invention, specifically using system 200. A similar workflow of some aspects of the method could be executed by system 100. Specifically, the program code is executed by a processing resource and obtains a predefined communication from an external communication device (S910). Based on obtaining the predefined communication, the program code executed by a processing resource activates a vibrating mechanism 220 (S920). When the vibrating mechanism 220 is activated and starts to vibrate, it causes the rotating mechanism 222 to spin (S930). As the rotating mechanism 222 spins, the activation arm 282 moves with the rotating mechanism 222 (S940). The movement of the activation arm 282 may in turn cause the connecting member 286 to move (S950). As the connecting member 286 moves the latch 290 will also translate (S960). The translation of the latch 290 will cause the latch 290 to disengage from the spring 262 (S970). Once the latch 290 releases the spring 262, the spring 262, which is coupled to the injection mechanism 260, exerts a force on the injection mechanism 260 propelling the injection mechanism 260 into the patient (S980). Concurrently, the program code executed by the processor starts the pump 240 (S925). When the pump 240 is activated, fluid or medication from the reservoir 250 is pumped to the injection mechanism 260 (S935). The fluid or medication travels from the reservoir 250 by the fluid pathway 270 through the pump 240 to the injection mechanism 260 for delivery to the patient. Once the requested amount of fluid or medication from the reservoir 250 is delivered to the patient, program code executed by a processor and sends a control signal to shut off the pump 240 and sends a control signal to extract the injection mechanism 260 from the deployed position (S990). When the injection mechanism 260 is extracted from the deployed position, the spring 262 is again engaged by the latch 290 to secure the injection mechanism 260 in a resting or undeployed position (S995).

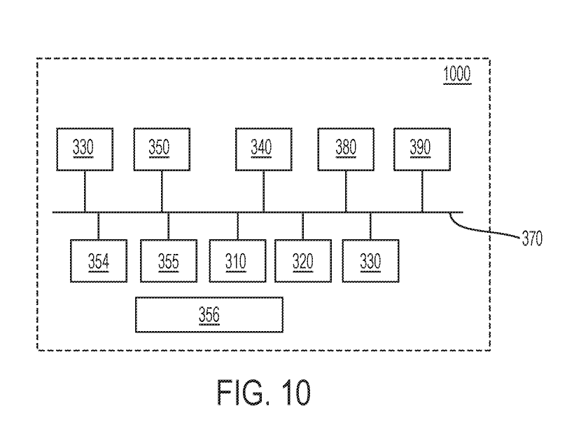

FIG. 10 is a component-level diagram of one embodiment of an EIR terminal 1000 that can be integrated into an embodiment of the described systems 100, 200. In an embodiment of the present systems 100, 200, the EIR terminal 1000 may comprise at least one microprocessor 310 and a memory 320, both coupled to the system bus 370. The microprocessor 310 can be provided by a general purpose microprocessor or by a specialized microprocessor (e.g., an ASIC). In one embodiment, EIR terminal 1000 can comprise a single microprocessor which can be referred to as a central processing unit (CPU). In another embodiment, EIR terminal 1000 can comprise two or more microprocessors, for example, a CPU providing some or most of the EIR terminal 1000 functionality and a specialized microprocessor performing some specific functionality. A skilled artisan would appreciate the fact that other schemes of processing tasks distribution among two or more microprocessors are within the scope of this disclosure.

EIR terminal 1000 can further comprise a communication interface 340 communicatively coupled to the system bus 370. In one embodiment, the communication interface can be provided by a wireless communication interface. The wireless communication interface can be configured to support, for example, but not limited to, the following protocols: at least one protocol of the IEEE 802.11/802.15/802.16 protocol family, at least one protocol of the HSPA/GSM/GPRS/EDGE protocol family, TDMA protocol, UMTS protocol, LTE protocol, and/or at least one protocol of the CDMA/1×EV-DO protocol family.

EIR terminal 1000 can further comprise a keyboard interface 354 and a display adapter 355, both also coupled to the system bus 370. EIR terminal 1000 can further comprise a battery 356. In one embodiment, the battery 356 can be provided by a replaceable rechargeable battery pack.

EIR terminal 1000 can further comprise a GPS receiver 380. EIR terminal 1000 can further comprise at least one connector 390 configured to receive a subscriber identity module (SIM) card.

EIR terminal 1000 can further comprise one or more EIR devices 330, provided, for example, but not limited to, by an RFID reading device, a bar code reading device, or a card reading device. In one embodiment, the EIR terminal 1000 can be configured to read an encoded message using EIR device 330, such as the label on the reservoir 150, 250 and to output raw message data containing the encoded message, for example, to send a communication including this information to the computing device that originally activated the system 100, 200. In another embodiment, the EIR terminal 1000 can be configured to read an encoded message using EIR device 330, and to output decoded message data corresponding to the encoded message. As used herein, “message” is intended to denote a character string comprising alphanumeric and/or non-alphanumeric characters. An encoded message can be used to convey information, such as identification of the source and the model of a product, for example, in a UPC code.

Mobile computing devices that read bar codes, read RFID, or read cards bearing encoded information may read more than one of these categories while remaining within the scope of this disclosure. For example, a device that reads bar codes may include a card reader, and/or RFID reader; a device that reads RFID may also be able to read bar codes and/or cards; and a device that reads cards may be able to also read bar codes and/or RFID.

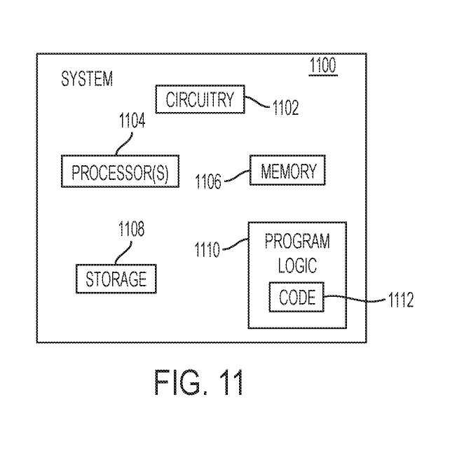

FIG. 11 illustrates a block diagram of a resource 1100, like communication device 294, external server 292, and controller 130, 230, which is part of the technical architecture of certain embodiments of the technique. The resource 1100 may include a circuitry 1102 that may in certain embodiments include a microprocessor 1104. The computer system 1100 may also include a memory 1106 (e.g., a volatile memory device), and storage 1108. The storage 1108 may include a non-volatile memory device (e.g., EEPROM, ROM, PROM, RAM, DRAM, SRAM, flash, firmware, programmable logic, etc.), magnetic disk drive, optical disk drive, tape drive, etc. The storage 208 may comprise an internal storage device, an attached storage device and/or a network accessible storage device. The system 1100 may include a program logic 1110 including code 1112 that may be loaded into the memory 1106 and executed by the microprocessor 1104 or circuitry 1102.

In certain embodiments, the program logic 1110 including code 1112 may be stored in the storage 1108 or memory 1106. In certain other embodiments, the program logic 1110 may be implemented in the circuitry 1102. Therefore, while FIG. 11 shows the program logic 1110 separately from the other elements, the program logic 1110 may be implemented in the memory 1106 and/or the circuitry 1102.

Using the processing resources of a resource 1100 to execute software, computer-readable code or instructions, does not limit where this code can be stored. The terms program logic, code, and software are used interchangeably throughout this application.

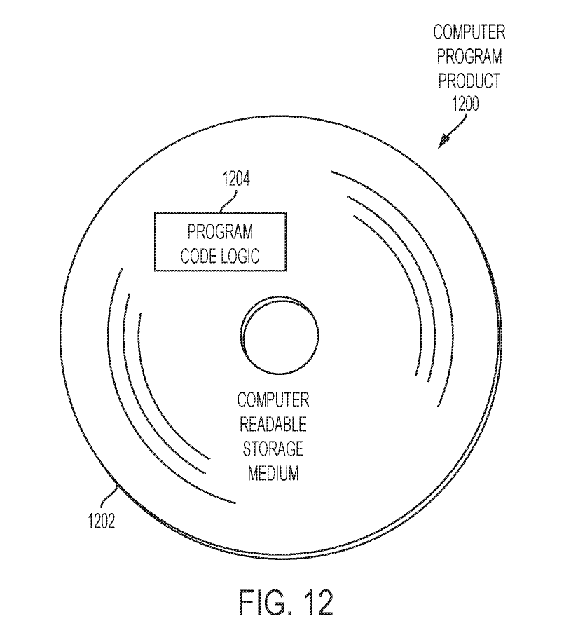

Referring to FIG. 12, in one example, a computer program product 1200 includes, for instance, one or more non-transitory computer readable storage media 1202 to store computer readable program code means or logic 1204 thereon to provide and facilitate one or more aspects of the technique.

As will be appreciated by one skilled in the art, aspects of the technique may be embodied as a system, method or computer program product. Accordingly, aspects of the technique may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the technique may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Program code embodied on a computer readable medium may be transmitted using an appropriate medium, including but not limited to wireless, wireline, optical fiber cable, RF, etc., or any suitable combination of the foregoing.

Computer program code for carrying out operations for aspects of the technique may be written in any combination of one or more programming languages, including an object oriented programming language, such as Java, Smalltalk, C++ or the like, and conventional procedural programming languages, such as the “C” programming language, assembler or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the technique are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions, also referred to as computer program code, may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the technique. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In addition to the above, one or more aspects of the technique may be provided, offered, deployed, managed, serviced, etc. by a service provider who offers management of customer environments. For instance, the service provider can create, maintain, support, etc. computer code and/or a computer infrastructure that performs one or more aspects of the technique for one or more customers. In return, the service provider may receive payment from the customer under a subscription and/or fee agreement, as examples. Additionally or alternatively, the service provider may receive payment from the sale of advertising content to one or more third parties.

In one aspect of the technique, an application may be deployed for performing one or more aspects of the technique. As one example, the deploying of an application comprises providing computer infrastructure operable to perform one or more aspects of the technique.

As a further aspect of the technique, a computing infrastructure may be deployed comprising integrating computer readable code into a computing system, in which the code in combination with the computing system is capable of performing one or more aspects of the technique. As a further aspect of the technique, the system can operate in a peer to peer mode where certain system resources, including but not limited to, one or more databases, is/are shared, but the program code executable by one or more processors is loaded locally on each computer, including the controller 130, 230.

As yet a further aspect of the technique, a process for integrating computing infrastructure comprising integrating computer readable code into a computer system may be provided. The computer system comprises a computer readable medium, in which the computer medium comprises one or more aspects of the technique. The code in combination with the computer system is capable of performing one or more aspects of the technique.

Further, other types of computing environments can benefit from one or more aspects of the technique. As an example, an environment may include an emulator (e.g., software or other emulation mechanisms), in which a particular architecture (including, for instance, instruction execution, architected functions, such as address translation, and architected registers) or a subset thereof is emulated (e.g., on a native computer system having a processor and memory). In such an environment, one or more emulation functions of the emulator can implement one or more aspects of the technique, even though a computer executing the emulator may have a different architecture than the capabilities being emulated. As one example, in emulation mode, the specific instruction or operation being emulated is decoded, and an appropriate emulation function is built to implement the individual instruction or operation.

In an emulation environment, a host computer includes, for instance, a memory to store instructions and data; an instruction fetch unit to fetch instructions from memory and to optionally, provide local buffering for the fetched instruction; an instruction decode unit to receive the fetched instructions and to determine the type of instructions that have been fetched; and an instruction execution unit to execute the instructions. Execution may include loading data into a register from memory; storing data back to memory from a register; or performing some type of arithmetic or logical operation, as determined by the decode unit. In one example, each unit is implemented in software. For instance, the operations being performed by the units are implemented as one or more subroutines within emulator software.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A drug delivery system, comprising:
an activation module;
a vibrating mechanism electrically coupled to the activation module;
a pumping mechanism connected to the activation module;
a reservoir; and
an injection mechanism coupled to the reservoir by at least one fluid pathway;
wherein the at least one fluid pathway extends through the pumping mechanism.

2. The drug delivery system of claim 1, wherein the vibrating mechanism is positioned on top of the pumping mechanism.

3. The drug delivery system of claim 1, wherein the vibrating mechanism is positioned next to the activation module and coupled to the injection mechanism by an actuation mechanism.

4. The drug delivery system of claim 1, wherein the pumping mechanism comprises:
a membrane positioned under the vibrating mechanism.

5. The drug delivery system of claim 1, wherein the pumping mechanism comprises:
a rotary pump positioned between the reservoir and the injection mechanism and in fluid communication with the at least one fluid pathway.

6. The drug delivery system of claim 1, wherein the pumping mechanism comprises:
a peristaltic pump positioned along the at least one fluid pathway between the reservoir and the injection mechanism.

7. The drug delivery system of claim 1, wherein the reservoir comprises a means for releasing pressure.

8. The drug delivery system of claim 7, wherein the reservoir is selected from a rigid container and a flexible container.

9. The drug delivery system of claim 1, further comprising:
at least one sensor electrically coupled to the activation module.

10. The activation module of claim 1, wherein the activation module comprises:
a receiver for receiving an activation signal to start the drug delivery system.

11. A vibratory drive mechanism, comprising:
an activation module;
a vibrating mechanism electrically coupled to the activation module; and
an actuation mechanism coupled to the vibrating mechanism.

12. The vibratory drive mechanism of claim 1, further comprising:
a receiver coupled to the activation module for remotely activating the vibratory drive mechanism.

13. The vibratory drive mechanism of claim 12, wherein the actuation mechanism comprises:
a pumping membrane in direct contact with the vibrating mechanism.

14. The vibratory drive mechanism of claim 12, wherein the activation mechanism comprises:
an activation arm with a first end and a second end, the first end coupled to the vibrating mechanism;
a connecting member with a first end and a second end, the first end of the connecting member rotatably coupled to the second end of the activation arm; and
a latch with a first end and a second end, the first end of the latch rotatably coupled to the second end of the connecting member.

15. A method of using a remotely activated drug delivery system, comprising:
positioning the remotely activated drug delivery system on a patient;
sending an activation signal to the remotely activated drug delivery system to administer an injection;
receiving the activation signal in the remotely activated drug delivery system;
processing the activation signal to deploy an injection mechanism of the remotely activated drug delivery system to deliver a medication to the patient; and
retracting the injection mechanism after the medication is delivered to the patient.

16. The method of claim 15, wherein the remotely activated drug delivery system comprises:
an activation module;
a vibrating mechanism coupled to the activation module;
a pumping mechanism connected to the activation module;
a reservoir; and
the injection mechanism coupled to the reservoir by at least one fluid pathway;
wherein the at least one fluid pathway extends through the pumping mechanism.

17. The method of claim 16, wherein processing the activation signal to deploy the injection mechanism of the remotely activated drug delivery system to deliver the medication to the patient, comprises:
sending a first signal from the activation module to the vibrating mechanism to commence vibrating;
sending a second signal from the activation module to start a motor to deploy the injection mechanism;
deploying the injection mechanism by rotating the motor; and
moving the medication from the reservoir through the at least one fluid pathway to the injection mechanism with the vibrations from the vibrating mechanism exerting a force on the pumping mechanism.

18. The method of claim 17, wherein the pumping mechanism is a pliable membrane positioned over the at least one fluid pathway and coupled to at least one wall of the remotely activated drug delivery system.

19. The method of claim 16, wherein processing the activation signal to deploy the injection mechanism of the remotely activated drug delivery system to deliver the medication to the patient, comprises:
sending a first signal from the activation module to the vibrating mechanism to commence vibrating to deploy the injection mechanism;
sending a second signal from the activation module to the pumping mechanism to start pumping the medication from the reservoir;
deploying the injection mechanism by releasing an actuation mechanism, the actuation mechanism being coupled to the vibrating mechanism; and
pumping the medication from the reservoir through the at least one fluid pathway to the injection mechanism with the pumping mechanism.

20. The method of claim 19, wherein the actuation mechanism comprises:
an activation arm with a first end and a second end, the first end coupled to the vibrating mechanism;
a connecting member with a first end and a second end, the first end of the connecting member rotatably coupled to the second end of the activation arm;
a latch with a first end and a second end, the first end of the latch rotatably coupled to the second end of the connecting member; and
wherein the second end of the latch engages a spring coupled to the injection mechanism.
